(19) United States
(12) Patent Application Publication
(10) Pub. No.: US 2014/0275148 A1
(43) Pub. Date: Sep. 18, 2014

(54) ORALLY ADMINISTRABLE, SELF-SUPPORTING DISSOLVING FILM DOSAGE FORMS

(71) Applicant: Novus Pharma LLC, Valley Cottage, NY (US)

(72) Inventors: Manish S. SHAH, West Caldwell, NJ (US); Ray J. DiFalco, Ridgewood, NJ (US)

(73) Assignee: Novus Pharma LLC, Valley Cottage, NY (US)

(21) Appl. No.: 14/218,687

(22) Filed: Mar. 18, 2014

Related U.S. Application Data
(60) Provisional application No. 61/798,617, filed on Mar. 15, 2013.

(51) Int. Cl. A61K 31/485; A61K 47/32; A61K 9/70

(52) U.S. Cl. CPC A61K 31/485; A61K 9/7007; A61K 47/32
USPC 514/282

(57) ABSTRACT

Orally administrable, self-supporting, dissolving film dosage forms comprising an active ingredient and a polyvinyl alcohol-polyethylene glycol graft copolymer, and methods of orally administering the film dosage forms are provided.

### CROSS-REFERENCE TO RELATED APPLICATION

This application claims the benefit of U.S. Patent Provisional Application No. 61/798,617, filed on Mar. 15, 2013, which is incorporated herein by reference.

### FIELD OF THE INVENTION

The invention relates to orally administrable, self-supporting, dissolving film dosage forms comprising an active ingredient and a polyvinyl alcohol-polyethylene glycol graft copolymer, and methods of orally administering the film dosage forms.

### BACKGROUND OF THE INVENTION

Active ingredients can be administered in a number of different types of oral dosage forms. Tablets and capsule dosage forms are typically used. However, such dosage forms have certain disadvantages. For example, tablets and capsules are often not ideal for individuals who have difficulty swallowing. Dissolving compositions, such as troches, wafers, or film compositions, are often an alternative to traditional tablet and capsule dosage forms. Foam wafers, which are typically solidified foams containing spaces or cavities and having an uneven or irregular surface, are described in U.S. Patent Application Publication No. 2009/0087486. Film dosage forms are typically in the form of strips having a relatively even surface and lacking any large spaces or cavities. Film dosage forms can be administered to mucosal membranes, including the mouth, rectum, vaginal, nasal and ears. Orally administrable self-supporting film dosage forms can be administered by placement in the mouth of a subject, for example, sublingually, and the film dosage form dissolves in the mouth.

Film dosage forms are often individually packaged. Individually packaged film dosage forms are often more difficult to tamper with, compared to traditional packages for tablets and capsules, such as bottles or foil blister packs. Therefore, film dosage forms of abused drugs, such as opioids, are beneficial, as it is more difficult for children or abusers to tamper with and take large amounts of these drugs in a short period of time to attain a “high.”

The present invention provides a dissolving film dosage form which can be administered conveniently without requiring water for ingestion, which can be dissolved rapidly, which has a decreased potential for the patient to expel the dosage form after administration and placement in the oral cavity, and which can result in improved patient compliance.

All references cited herein are hereby incorporated by reference in their entirety.

### SUMMARY OF THE INVENTION

The present invention provides an orally administrable, self-supporting, dissolving film dosage form comprising an active ingredient and a polyvinyl alcohol-polyethylene glycol graft copolymer. The present invention also provides methods of treating or reducing symptoms associated with a medical condition, comprising administering the film dosage form to a subject in need thereof.

### DETAILED DESCRIPTION OF THE INVENTION

The present invention provides an orally administrable, self-supporting, dissolving film dosage form comprising an active ingredient. In some embodiments, the dosage form comprises more than one active ingredient.

The term “active ingredient” includes any compound or drug which has pharmacological, therapeutic, or biological activity. In some embodiments, the active ingredient include, but are not limited to analgesics, anti-inflammatory agents, anti-helminthics, anti-arrhythmic agents, anti-asthma agents, anti-bacterial agents, anti-viral agents, anti-coagulants, anti-dementia agents, anti-depressants, anti-diabetics, anti-epileptics, anti-fungal agents, anti-gout agents, anti-hypertensive agents, anti-malarials, anti-migraine agents, anti-muscarinic agents, anti-neoplastic agents, immunosuppressants, anti-protozoal agents, anti-thyroid agents, anti-tussives, anxiolytics, sedatives, hypnotics, neuroleptics, neuroprotective agents, β-blockers, cardic inotropic agents, cell adhesion inhibitors, corticosteroids, cytokine receptor activity modulators, diuretics, anti-Parkinson's agents, gastro-intestinal agents, histamine H-receptor antagonists, keratolytics, lipid regulating agents, muscle relaxants, nitrates and other anti-anginal agents, non-steroid anti-asthma agents, nutritional agents, opioid analgesics, sex hormones, stimulants and anti-erectile dysfunction agents; and salts, esters, and mixtures thereof. In preferred embodiments, the active ingredient comprises a drug which is often abused, such as a central nervous system stimulant or depressant. Examples of central nervous system stimulants include, but are not limited to, amphetamines and agents such as cocaine. Examples of central nervous depressants include, but are not limited to but are not limited to opioids, barbiturates, benzodiazepines, and other anxiety and sleep medications.

Stimulants increase heart rate, blood pressure and metabolism, sometimes providing feelings of exhilaration and energy and increased mental alertness. Amphetamines such as methylphenidate (sometimes marketed under the tradename RITALIN®) dextroamphetamine (sometimes marketed under the tradenames ADDERALL® and DEXEDRINE®), modafinil (PROVIGIL®), and dexmethylphenidate (FOCALIN®) are often prescribed for the treatment of narcolepsy, attention-deficit/hyperactivity disorder, and depression that has not responded to other treatments. They also may be used for short-term treatment of obesity. Individuals may become addicted to the sense of well-being and enhanced energy that stimulants can generate. Taking high doses of stimulants repeatedly over a short time, however, can lead to feelings of hostility or paranoia. Additionally, taking high doses of stimulants may result in dangerously high body temperatures and an irregular heartbeat.

Examples of opioids and opioid antagonists include, but are not limited to the following: alfentanil, allylprodine, alphaprodine, anileridine, benzylmorphine, bezitramide, buprenorphine, butorphanol, clonitazene, codeine, desomorphine, dextromoramide, dezocine, diampromide, diamorphone, dihydrocodeine, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazene, hydrocodone, hydromorphone, hydroxypethidine, isomethadone, ketobemidone, levorphanol, levophenacylmorphan, lofentanil, meperidine, meptazinol, metazocine, methadone, metopon, morphine, myrophine, narceine, naloxone, naltrexone, nicomorphine, norlevorphanol, normethadone, nalorphine, nalbuphene, normorphine, norpipanone, opium, oxycodone, oxymorphone, papaveretum, pentazocine, phenadoxone, phenomorphan, phenazocine, phenoperidine, piminodine, piritramide, propheptazine, promedol, properidine, propoxyphene, sufentanil, tilidine, and tramadol. Any opioid or opioid antagonist or pharmaceutically acceptable salt or ester thereof may be used in the film dosage form.

Preferred opioids include fentanyl, sufentanil, carfentanil, lofentanil, alfentanil, hydromorphone, oxycodone, morphine, hydroxycodone, propoxyphene, pentazocine, methadone, tilidine, butorphanol, buprenorphine, levorphanol, codeine, oxymorphone, meperidine, dihydrocodeinone. Preferred opioid antagonists include naloxone and naltrexone.

Examples of barbiturates include, but are not limited to mephobarbital (which is sometimes marketed under the tradename MEBARAL®) and pentobarbital sodium (which is sometimes marketed under the tradename NEMBUTAL®). Barbiturates are often prescribed to treat anxiety, tension, and sleep disorders.

Examples of benzodiazepines and benzodiazepine derivatives include, but are not limited to diazepam (sometimes marketed under the tradename VALIUM®), alprazolam (sometimes marketed under the tradename XANAX®), triazolam (HALCION®), and estazolam (PROSOM®). Other example include: chlordiazepoxide, temazepam, lorazepam, clonazepam, flurazepam, midazolam, and oxazepam. Benzodiazepines are often prescribed to treat anxiety, acute stress reactions, and panic attacks.

Other CNS depressants include zaleplon, which is sometimes marketed under the trade name SONATA® and zolpidem, which is sometimes marketed under the trade name AMBIEN®.

In some embodiments, the film dosage form comprises an analgesic such as an opioid. In some embodiments, the opioid is selected from the group consisting of: buprenorphine, oxycodone, hydrocodone, oxymorphone, hydromorphone, codeine, and methadone or salts thereof. In some preferred embodiments, the film dosage form comprises buprenorphine or a salt thereof. In some embodiments, the film dosage form comprises an opioid antagonist. In some preferred embodiments, the opioid antagonist is selected from the group consisting of naloxone and naltrexone or salts thereof.

In some embodiments, the film dosage form comprises more than one drug. In some embodiments, the film dosage form comprises more than one opioid, or an opioid and an opioid antagonist. In some preferred embodiments, the film dosage form comprises buprenorphine or a salt thereof, such as buprenorphine hydrochloride, and naloxone or a salt thereof, such as naloxone hydrochloride.

In some embodiments, the film comprises an opioid, such as buprenorphine or a salt thereof, and an opioid antagonist, such as naloxone or a salt thereof, in a weight ratio of opioid:opioid antagonist of between about 10:1 to 1:10, preferably about 6:1 to 1:1, more preferably about 5:1 to 3:1, even more preferably about 4:1 to 3:1. In some embodiments, the weight ratio of opioid:opioid antagonist is about 4:1 to 3:5:1.

In some embodiments the film dosage form comprises buprenorphine or a salt thereof in an amount of between about 1 mg to about 20 mg, alternatively between about 2 mg to about 12 mg, alternatively about 2 mg to about 10 mg, alternatively about 2 mg to about 8 mg. In some embodiments, the film dosage form comprises naloxone or a salt thereof in an amount of between about 0.25 mg and about 10 mg, alternatively about 0.5 mg to about 8 mg, alternatively about 0.5 mg to about 4 mg, alternatively about 0.5 mg to about 3 mg.

In some embodiments, the film dosage form comprises the film dosage form comprises about 2 mg of buprenorphine or a salt thereof and about 0.5 mg of naloxone or a salt thereof. In some embodiments, the film dosage form comprises about 4 mg of buprenorphine or a salt thereof and about 1 mg of naloxone or a salt thereof. In some embodiments, the film dosage form comprises about 8 mg of buprenorphine or a salt thereof and about 2 mg of naloxone or a salt thereof. In some embodiments, the film dosage form comprises about 12 mg of buprenorphine or a salt thereof and about 3 mg of naloxone or a salt thereof.

The term “dissolving film dosage form” refers to a dosage form in the form of a thin sheet or film that can be administered orally to a subject, preferably a human subject. The dissolving film dosage form contains an active ingredient and one or more pharmaceutically acceptable excipients. The active ingredient in the film dosage form may be dissolved in solution or suspended in the film. Upon placement of the film dosage form in the mouth of a subject, the film dosage form dissolves, releasing the active ingredient, and making it available for absorption in the oral cavity. In preferred embodiments, substantially all of the film dosage form dissolves in the oral cavity. “Substantially all of the film dosage form” refers to more than about 50%, preferably more than about 75%, and more preferably more than about 90% of the film dosage form.

Examples of dissolving film dosage forms are described in U.S. Pat. Nos. 4,136,145, 4,849,246, 5,629,003, 5,948,430, 7,357,891; 7,425,292, and 8,017,150 which are each incorporated by reference in their entirety.

The self-supporting, dissolving film dosage form comprises a polyvinyl alcohol-polyethylene glycol graft copolymer. In preferred embodiments, the polyvinyl alcohol-polyethylene glycol graft copolymer is the polyvinyl alcohol-polyethylene glycol graft copolymer sold under the trade name KOLLICOAT® IR (BASF, Ludwigshafen, Germany), which contains about 75% polyvinyl alcohol units and about 25% polyethylene glycol units. In some embodiments, the polyvinyl alcohol-polyethylene glycol graft copolymer has a molecular weight of about 5,000 to about 100,000 Daltons, preferably about 10,000 to about 75,000 Daltons, more preferably about 20,000 to about 60,000 Daltons, even more preferably about 40,000 to about 50,000 Daltons, and most preferably about 45,000 Daltons. The polyvinyl alcohol-polyethylene glycol graft copolymer may form a polymer matrix, wherein the matrix contains the active ingredient(s).

In preferred embodiments, the dissolving film dosage form comprises polyvinyl alcohol-polyethylene glycol graft copolymer in an amount that ranges from about 10% to about 95%, preferably about 40% to about 93%, more preferably about 70% to about 90% by weight, and most preferably about 75% to about 85% of the dosage form. In some embodiments, the film dosage form may further comprise one or more additional film-forming polymers. The film-forming polymer may comprise a water-soluble polymer, a water-insoluble polymer, or a combination of one or more water-soluble polymer and/or water-insoluble polymers. Examples of further film-forming polymers include but are not limited to cellulose or cellulose derivative polymers and polyethylene oxide.

In some embodiments, the self-supporting, dissolving film dosage form has a uniform thickness. In some embodiments, the dissolving film dosage form has a thickness of about 1 μm to about 5 mm, preferably about 50 μm to about 3 mm, more preferably about 100 μm to about 2 mm, even more preferably about 250 μm to about 1 mm, even more preferably about 350 μm to about 750 μm, and most preferably about 500 μm. In some embodiments, the dissolving film dosage form is in a size that can comfortably be placed in the mouth. For example, in some embodiments, the film dosage form may have an area of about 0.5 cm² to about 5 cm², preferably about 1 cm² to about 4 cm², more preferably about 2 cm² to about 3 cm², and most preferably about 2.5 cm². In some preferred embodiments, the self-supporting, dissolving film dosage form has a smooth texture, wherein there are no significant spaces or cavities and there is minimal or no crystallization. In some embodiments, the dissolving film dosage form comprises the film forming polymer, such as polyvinyl alcohol-polyethylene glycol graft copolymer, in an amount greater than about 35 mg/cm², preferably about 36 mg/cm² to about 90 mg/cm², more preferably about 38 mg/cm² to about 75 mg/cm², even more preferably about 39 mg/cm² to about 50 mg/cm², even more preferably about 40 mg/cm² to about 50 mg/cm², and most preferably about 40 mg/cm² to about 45 mg/cm².

The self-supporting, dissolving film dosage form of the invention may comprise one or more pharmaceutically acceptable excipients. These may include, without limitation, surfactants; plasticizers which assist in compatibilizing the components within the mixture; polyalcohols; anti-foaming agents, such as silicone-containing compounds, which promote a smoother film surface by releasing oxygen from the film; and thermo-setting gels such as pectin, carageenan, and gelatin, which help in maintaining the dispersion of components.

The variety of pharmaceutically acceptable excipients that can be incorporated into the inventive compositions may provide a variety of different functions. Examples of classes of pharmaceutically acceptable excipients include lubricants, buffering agents, stabilizers, blowing agents, pigments, coloring agents, fillers, bulking agents, fragrances, release modifiers, adjuvants, plasticizers, flow accelerators, mold release agents, polyols, granulating agents, diluents, binders, buffers, absorbents, glidants, adhesives, anti-adherents, acidulants, softeners, resins, demulcents, solvents, surfactants, emulsifiers, elastomers and mixtures thereof. These additives may be added with the active ingredient(s).

Additional useful additives include, for example, gelatin, vegetable proteins such as sunflower protein, soybean proteins, cotton seed proteins, peanut proteins, grape seed proteins, whey proteins, whey protein isolates, blood proteins, egg proteins, acrylated proteins.

Further pharmaceutically acceptable excipients may be inorganic fillers, such as the oxides of magnesium aluminum, silicon, titanium, etc. desirably in a concentration range of about 0.02% to about 3% by weight and desirably about 0.02% to about 1% based on the weight of all components.

Further examples of pharmaceutically acceptable excipients are plasticizers which include polyalkylene oxides, such as polyethylene glycols, polypropylene glycols, polyethylene-propylene glycols, organic plasticizers with low molecular weights, such as glycerol, glycerol monoacetate, diacetate or triacetate, triacetin, polysorbate, cetyl alcohol, propylene glycol, sorbitol, sodium diethylsulfosuccinate, triethyl citrate, tribuaddittyl citrate, and the like, which, if present, may be added in concentrations ranging from about 0.5% to about 10% by weight of the dosage form.

There may be further added compounds to improve the flow properties of the starch material such as animal or vegetable fats, desirably in their hydrogenated form, especially those which are solid at room temperature. These fats desirably have a melting point of 50° C. or higher. Preferred are tri-glycerides with C₁₂, C₁₄, C₁₆, C₁₈, C₂₀ and C₂₂ fatty acids. These fats can be added alone without adding extenders or plasticizers and can be advantageously added alone or together with mono- and/or di-glycerides or phosphatides, especially lecithin. The mono- and di-glycerides are desirably derived from the types of fats described above, i.e. with C₁₂, C₁₄, C₁₆, C₁₈, C₂₀ and C₂₂ fatty acids. The total amounts used of the fats, mono-, di-glycerides and/or lecithins are up to about 5% and preferably within the range of about 0.5% to about 2% by weight of the total composition.

It is further useful to add texturizing agents including, but not limited to, silicon dioxide, calcium silicate, or titanium dioxide, preferably, when present, in a concentration of about 0.02% to about 1% by weight of the total composition.

Lecithin or other one surface active agents may be used in the present invention. The surface active agents, if present, may be included in the feedstock in an amount of from about 0.25% to about 2.00% by weight. Other surface active agents, i.e. surfactants, include, but are not limited to, cetyl alcohol, sodium lauryl sulfate, the Spans™ and Tweens™ which are commercially available from ICI Americas, Inc. Ethoxylated oils, including ethoxylated castor oils, such as Cremophor® EL which is commercially available from BASF, are also useful. Tweens™ or combinations of surface active agents may be used to achieve the desired hydrophilic-lipophilic balance (“HLB”). The present invention, however, does not require the use of a surfactant and films or film-forming compositions of the present invention may be essentially free of a surfactant while still providing the desirable uniformity features of the present invention.

Other ingredients include binders which contribute to the ease of formation and general quality of the films. Non-limiting examples of binders include starches, pregelatinize starches, gelatin, polyvinylpyrrolidone, methylcellulose, sodium carboxymethylcellulose, ethylcellulose, polyacrylamides, polyvinyloxoazolidone, and polyvinylalcohols.

Anti-foaming and/or de-foaming components may also be used with the films of the present invention. These components aid in the removal of air, such as entrapped air, from the film-forming compositions, which may lead to non-uniform films. Simethicone and silicone-containing compounds, such as silicone dioxide, are useful anti-foaming and/or de-foaming agents. The present invention, however, is not so limited and other anti-foam and/or de-foaming agents may suitable be used.

Buffering agents or pH adjusting agents may also be used, such as calcium carbonate, sodium bicarbonate, citric acid, sodium citrate anhydrous, tartaric acid, succinic acid, maleic acid, and fumaric acid.

Antioxidants and preservatives may also be added to the film. Examples of antioxidants and preservatives include, but are not limited to parabens, such as methyl paraben, ethyl paraben, propyl paraben, and butyl paraben, benzoic acid, sodium benzoate, sorbic acid, sodium sorbate, cetrimide, benzalkonium chlorise, cetylpyridium chloride, benzaethonium chloride, phenylmercuric nitrate, benzyl alcohol, phenylethyl alcohol, bronabol, chlorbutanol, chlorhexidine, butylated hydroxyanisole, butylated hydroxytoluene, tert-butyl hydroquinone, and 4-hydroxymethyl-2,6,-di-ter-butylphenol.

The film dosage form may also comprise coloring agents. Examples of coloring agents or additives include, but are not limited to food, drug and cosmetic colors (FD&C), drug and cosmetic colors (D&C), or external drug and cosmetic colors (Ext. D&C). These colors are dyes, their corresponding lakes, and certain natural and derived colorants. Lakes are dyes absorbed on aluminum hydroxide. Other examples of coloring agents include known azo dyes, organic or inorganic pigments, or coloring agents of natural origin. Inorganic pigments are preferred, such as the oxides or iron or titanium, these oxides, being added in concentrations ranging from about 0.001 to about 10%, and preferably about 0.5 to about 3%, based on the weight of all the components.

Flavors may be chosen from natural and synthetic flavoring liquids. Examples of flavoring agents include, but are not limited to, volatile oils, synthetic flavor oils, flavoring aromatics, oils, liquids, oleoresins or extracts derived from plants, leaves, flowers, fruits, stems and combinations thereof. A non-limiting representative list of examples includes mint oils, cocoa, and citrus oils such as lemon, orange, grape, lime and grapefruit and fruit essences including apple, pear, peach, grape, strawberry, raspberry, cherry, plum, pineapple, apricot or other fruit flavors. Other useful flavoring agents include aldehydes and esters such as benzaldehyde (cherry, almond), citral i.e., alphacitral (lemon, lime), neral, i.e., beta-citral (lemon, lime), decanal (orange, lemon), aldehyde C-8 (citrus fruits), aldehyde C-9 (citrus fruits), aldehyde C-12 (citrus fruits), tolyl aldehyde (cherry, almond), 2,6-dimethyloctanol (green fruit), and 2-dodecenal (citrus, mandarin), combinations thereof and the like.

The sweeteners may be chosen from the following non-limiting list: glucose (corn syrup), dextrose, invert sugar, fructose, and combinations thereof; saccharin and its various salts such as the sodium salt; dipeptide sweeteners such as aspartame; dihydrochalcone compounds, glycyrrhizin; Stevia Rebaudiana (Stevioside); chloro derivatives of sucrose such as sucralose; sugar alcohols such as sorbitol, mannitol, maltitol, xylitol, and erythritol. Also contemplated are hydrogenated starch hydrolysates and the synthetic sweetener 3,6-dihydro-6-methyl-1-1-1,2,3-o-xathiazin-4-one-2,2-dioxide, particularly the potassium salt (acesulfame potassium), ammoniated glycyrrhizin and monoammonium glycyrrhizinate, and sodium and calcium salts thereof, and natural intensive sweeteners. Other sweeteners may also be used.

The self-supporting, dissolving film dosage may be produced by any known methods in the art, such as those described in U.S. Pat. Nos. 4,631,837, 7,357,891; and 8,017,150, each of which are incorporated by reference. In some embodiments, the method of making the dissolving film dosage form of the present invention comprises: (1) preparing a mixture containing the active ingredient, polyvinyl alcohol-polyethylene glycol graft copolymer and water, (2) adding to the mixture one or more pharmaceutically acceptable excipients, such as sweeteners, flavoring agents, coloring agents and buffering agents, and (3) spreading the resulting mixture onto a coating support to form a film by drying. Methods of coating or casting to form a film dosage form include, but are not limited to roll coating, gravure coating, immersion or dip coating, metering rod or Meyer Bar coating, slot die or extrusion coating, gap or knife-over roll coating, air-knife coating, curtain coating, or combinations thereof.

The present invention also provides for methods of using the dissolving film dosage forms. In preferred embodiments, the dissolving film dosage form is placed in the oral cavity of the subject, such as on or under the tongue, and allowed to dissolve completely. The dissolving film dosage form may be administered to a subject in a fed state or a fasted state. The dissolving film dosage form may also be administered with or without the administration of water. In some embodiments, more than one dissolving film dosage form may be administered sequentially within a single dosage administration. When more than one dissolving film dosage form is administered sequentially, preferably the subject places the dissolving film dosage form in the oral cavity and allows the dosage form to completely dissolve before administration of the next dosage form.

The present invention provides methods of treating a disease or condition in a subject in need therefore, comprising administering to the subject the film dosage form. In some embodiments, the disease or condition is selected from the group consisting of: pain, opioid addiction, detoxification, and long term replacement in therapy for opioid dependency.

The self-supporting, dissolving film dosage forms may be administered to any subject, adult or pediatric, for any use which benefits from the administration of the active ingredient contained in the dosage form. For example, the dissolving film dosage form of the present invention may comprise buprenorphine and naloxone, and the dosage form may be suitable for treatment of opioid dependence or addiction.

The self-supporting, dissolving film dosage form may be administered to a subject at any dosing frequency. In some embodiments, the dosage form is administered 1 to 10 times daily. In some embodiments, wherein the dissolving film dosage form comprises buprenorphine and naloxone and is used for the treatment of opioid dependence or addiction, the dosage form may be administered 1 to 3 times daily, preferably 1 to 2 times daily, and most preferably 1 time daily.

### EXAMPLES

### Example 1

| Ingredient | mg/film strip | |
|---|---|---|
| Burprenorphine hydrochloride | 8 | mg |
| Naloxone hydrochloride | 2 | mg |
| Polyvinyl alcohol-polyethylene | 66.7 | mg |
| glycol graft copolymer | | |
| (KOLLICOAT® IR) | | |
| Maltitol | 10.0 | mg |
| Acesulfame potassium | 1.0 | mg |
| Lime flavor | 0.35 | mg |
| FD&C Yellow | 0.21 | mg |
| Citric acid | 0.7 | mg |

### Example 2

| Ingredient | mg/film strip | |
|---|---|---|
| Burprenorphine hydrochloride | 8 | mg |
| Naloxone hydrochloride | 2 | mg |
| Polyvinyl alcohol-polyethylene | 76.7 | mg |
| glycol graft copolymer | | |
| (KOLLICOAT® IR) | | |
| Acesulfame potassium | 1.00 | mg |
| Lime flavor | 0.35 | mg |
| FD&C Yellow | 0.21 | mg |
| Citric acid | 0.70 | mg |

### Example 3

| Ingredients | mg/film strip |
|---|---|
| Buprenorphine hydrochloride | 8 |
| Naloxone hydrochloride dihydrate | 2 |
| Polyvinyl alcohol polyethylene | 52.14 |
| glycol graft copolymer | |
| (KOLLICOAT® IR) | |
| Maltitol | 5.21 |
| Asesulfum potassium | 0.5 |
| Lime flavour | 0.1 |
| FD&C yellow | 0.13 |
| Citric acid | 0.5 |
| Total weight | 69.66 |

### Example 4

| Ingredients | mg/film strip |
|---|---|
| Buprenorphine hydrochloride | 2 |
| Naloxone hydrochloride dihydrate | 0.5 |
| Polyvinyl alcohol polyethylene | 52.14 |
| glycol graft copolymer | |
| Maltitol | 5.21 |
| Asesulfum potassium | 0.5 |
| Lime flavour | 0.1 |
| FD&C yellow | 0.11 |
| Citric acid | 0.5 |
| Total weight | 61.39 |

### Example 5

The following depicts the results of an experiment conducted to determine the physical stability of four formulations. The formulations were placed on a glass plate having an area of about 294 cm². The physical stability was assessed by determining whether there was any significant crystallization. Dosage forms comprising a larger amount of polyvinyl alcohol-polyethylene glycol graft copolymer (KOLLICOAT® IR) was determined to have better physical stability and less crystallization was observed.

| Ingredient No. | Experiment 1 | | Experiment 2 | | Experiment 3 | | Experiment 4 | |
|---|---|---|---|---|---|---|---|---|
| Buprenorphine | 1008.3 | mg | 1008.3 | mg | 1008.3 | mg | 1008.3 | mg |
| HCL | | | | | | | | |
| Naloxone HCL | 284.7 | mg | 284.7 | mg | 284.7 | mg | 284.7 | mg |
| KOLLICOAT® | 8000 | mg | 10000 | mg | 12000 | mg | 13000 | mg |
| IR | | | | | | | | |
| KOLLICOAT® | 27.2 | | 34.00 | | 40.80 | | 44.20 | |
| IR | | | | | | | | |
| (mg/cm²) | | | | | | | | |
| Acesulfame | 120 | mg | 120 | mg | 120 | mg | 120 | mg |
| Potassium | | | | | | | | |
| Citric acid | 60 | mg | 60 | mg | 60 | mg | 60 | mg |
| Sweetpearl | 25 | mg | 25 | mg | 25 | mg | 25 | mg |
| (maltitol) | | | | | | | | |
| Lemon Flavour | 12 | mg | 12 | mg | 12 | mg | 12 | mg |
| FD&C Colour | 1.6 | mg | 1.6 | mg | 1.6 | mg | 1.6 | mg |

| | | | | |
|---|---|---|---|---|
| Observations | Crystallization | Less than | Smooth film - | Smooth film - |
| | occur in film | 5% crystallization | no | no |
| | | | crystallization | crystallization |

What is claimed:

1. An orally administrable, self-supporting, dissolving film dosage form comprising an active ingredient and a polyvinyl alcohol-polyethylene glycol graft copolymer.

2. The film dosage form of claim 1, wherein the active ingredient is selected from the group consisting of: central nervous system stimulants and central nervous system depressants.

3. The film dosage form of claim 1, wherein the active ingredient comprises an opioid.

4. The film dosage form of claim 1, further comprising an additional active ingredient.

5. The film dosage form of claim 4, wherein the film dosage form comprises buprenorphine or a salt thereof and naloxone or a salt thereof.

6. The film dosage form of claim 5, wherein the film dosage form comprises buprenorphine or a salt thereof and naloxone or a salt thereof in a weight ratio of buprenorphone or a salt thereof:naloxone or a salt thereof of about 4:1.

7. The film dosage form of claim 5, comprising about 8 mg of buprenorphine or a salt thereof and about 2 mg of naloxone or a salt thereof.

8. The film dosage form of claim 1, further comprising one or more pharmaceutically active ingredients selected from the group consisting of: sweeteners, flavoring agents, coloring agents, and buffering agents.

9. The film dosage form of claim 8, wherein the dosage form comprises acesulfame potassium.

10. The film dosage form of claim 8, wherein the dosage form comprises citric acid.

11. The film dosage form of claim 8, wherein the dosage form comprises sodium citrate, anhydrous.

12. The film dosage form of claim 8, wherein the dosage form comprises maltitol.

13. The film dosage form of claim 1, wherein the dosage form comprises polyvinyl alcohol-polyethylene glycol graft copolymer in an amount greater than about 35 mg/cm².

14. The film dosage form of claim 1, wherein the dosage form comprises polyvinyl alcohol-polyethylene glycol graft copolymer in an amount of about about 39 mg/cm² to 50 mg/cm².

15. The film dosage form of claim 1, wherein the dosage form comprises polyvinyl alcohol-polyethylene glycol graft copolymer in an amount of about 40 mg/cm² to 50 mg/cm².

16. The film dosage form of claim 1, wherein the dosage form comprises polyvinyl alcohol-polyethylene glycol graft copolymer in an amount of about 40 mg/cm² to about 45 mg/cm².
